(19) United States
(12) Patent Application Publication
(10) Pub. No.: US 2022/0134056 A1
(43) Pub. Date: May 5, 2022

(54) VACUUM-INSULATED MEDICAL DEVICES

(71) Applicant: CONCEPT GROUP LLC, Wellestey, MA (US)

(72) Inventors: Peter ROACH, Jacksonville, FL (US); Shriram RADHAKRISHNAN, West Palm Beach, FL (US)

(21) Appl. No.: 17/434,485

(22) PCT Filed: Feb. 27, 2020
(86) PCT No.: PCT/US2020/020173

§ 371 Date: Aug. 27, 2021

Related U.S. Application Data
(60) Provisional application No. 62/811,162, filed on Feb. 27, 2019.
(60) Provisional application No. 62/982,458, filed on Feb. 27, 2020.

(51) Int. Cl. A61M 25/01

(52) U.S. Cl. CPC A61M 25/0138; A61M 25/0136; A61M 2025/0004; A61M 25/0147; A61M 25/0155

(57) ABSTRACT

Provided are medical devices that comprise first and second tubes with a sealed insulating space formed therebetween, the devices being steerable and configured to deliver a fluid to a treatment site on or within a subject. The tubes of the devices can include corrugations so as to allow for bendability of the devices. Also provided are methods of using the disclosed devices.

### RELATED APPLICATIONS

The present application claims priority to and the benefit of U.S. patent application No. 62/811,162, “Vacuum-Insulated Medical Devices” (filed Feb. 27, 2019), and U.S. patent application No. 62/982,458, “Articulated Insulated Components” (filed Feb. 27, 2020), the entireties of which applications are incorporated herein by reference for any and all purposes.

### TECHNICAL FIELD

The present disclosure relates to the field of thermally-insulated medical devices.

### BACKGROUND

In certain medical applications—e.g., cryosurgery, thermal ablation, and the like—it is desirable to deliver fluid at a specified temperature to a particular location on or in a subject. Existing fluid delivery systems typically comprise a tube that is in fluid communication with the fluid source (e.g., heated saline, liquid nitrogen), with the tube typically comprising an amount of bulky insulation disposed about the tube so as to (1) maintain the temperature of the fluid in the tube while the fluid transits through the tube; and (2) protect the user from discomfort or injury that could be caused by the high or low temperature of the fluid.

The insulation of existing systems, however, can make the tube difficult to insert into and remove from the subject, and can also make the tube difficult to manipulate while inside of the subject. Additionally, existing insulated systems are challenging to steer once within the subject, and the insulating capabilities of existing systems are not necessarily adequate to handle certain fluids at certain temperatures, thereby limiting the capabilities of such existing systems. Accordingly, there is a long-felt need in the art for improved systems for delivering fluids on or into a subject.

In meeting these described long-felt needs, the present disclosure first provides thermally-insulated steerable devices, comprising: an elongate body, the elongate body defining a major axis, the elongate body having a distal end, and the elongate body having a selectively steerable portion, the elongate body comprising an first tube; the elongate body comprising an second tube disposed within the first tube, the second tube defining a lumen therein, the lumen having a major axis, and the second tube and first tube defining a sealed insulating space of reduced pressure therebetween; a handle comprising an actuator; and a linkage in communication with the actuator, the linkage configured to effect a deflection of a portion of the elongate body from the major axis, a rotation of a portion of the elongate body about the major axis, or both.

Also provided are methods comprising operating the linkage of a device according to the present disclosure so as to effect a deflection of a portion of the elongate body from the major axis, a rotation of a portion of the elongate body about the major axis, or both.

Further provided are methods, comprising communicating a fluid within a device according to the present disclosure.

Additionally provided are thermally-insulated steerable devices, comprising: an elongate body, the elongate body defining a major axis, the elongate body having a distal end, and the elongate body having a selectively steerable portion, the elongate body comprising an first tube; the elongate body comprising an second tube disposed within the first tube, the second tube defining a lumen therein, the lumen having a major axis, and the second tube and first tube defining a sealed insulating space of reduced pressure therebetween; and a linkage configured to effect a deflection of a portion of the elongate body from the major axis, a rotation of a portion of the elongate body about the major axis, or both.

Also provided are methods of inserting such devices into subjects, effecting deflection of a portion of the elongate body from the major axis, a rotation of a portion of the elongate body about the major axis, or both. Also provided are methods of delivering fluid on or into a subject using the disclosed devices.

Further provided are thermally-insulated steerable devices, comprising: an elongate body, the elongate body defining a major axis, the elongate body having a distal end, and the elongate body having a selectively steerable portion, the elongate body comprising an first tube; the elongate body comprising an second tube disposed within the first tube, the second tube defining a lumen therein, the lumen having a major axis, and the second tube and first tube defining a sealed insulating space of reduced pressure therebetween; and a linkage configured to effect a deflection of a portion of the elongate body from the major axis, a rotation of a portion of the elongate body about the major axis, or both.

Also provided are thermally-insulated steerable devices, comprising: a plurality of segments, a segment comprising an first tube; a segment comprising an second tube disposed within the first tube, the second tube defining a lumen therein, the lumen having a major axis, and the second tube and first tube defining a sealed insulating space of reduced pressure therebetween; and a linkage configured to effect relative motion of one segment relative to another segment.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIG. 1 provides a cutaway view of an exemplary device;

FIG. 2 provides an exterior view of an exemplary device;

FIG. 3 provides an exterior view of an exemplary device;

FIG. 4 provides an exterior view of an exemplary device;

FIG. 5 provides an exterior view of an exemplary device; and

FIG. 6 provides an exterior view of an exemplary device.

### DETAILED DESCRIPTION OF ILLUSTRATIVE EMBODIMENTS

The present disclosure may be understood more readily by reference to the following detailed description taken in connection with the accompanying figures and examples, which form a part of this disclosure. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention.

Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable, and it should be understood that steps may be performed in any order.

It is to be appreciated that certain features of the invention which are, for clarity, described herein in the context of separate embodiments, may also be provided in combination in a single embodiment. Conversely, various features of the invention that are, for brevity, described in the context of a single embodiment, may also be provided separately or in any subcombination. All documents cited herein are incorporated herein in their entireties for any and all purposes.

Further, reference to values stated in ranges include each and every value within that range. In addition, the term “comprising” should be understood as having its standard, open-ended meaning, but also as encompassing “consisting” as well. For example, a device that comprises Part A and Part B can include parts in addition to Part A and Part B, but can also be formed only from Part A and Part B.

### FIGURES

The attached figures are illustrative only and do not limit the scope of the present disclosure or the attached claims.

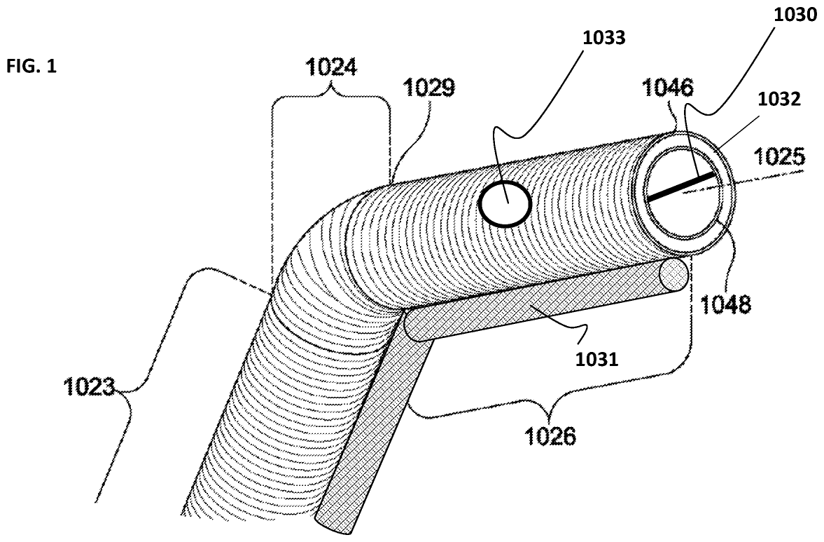

FIG. 1 provides a cutaway view of an exemplary device. As shown, a device can include a second tube 1048 disposed within a first tube 1046. (The centerline 1025 of the lumen of the second tube is shown for convenience.) The tubes can define sealed insulating space 1032 therebetween. Corrugations 1029 are shown on the exterior of first tube 1046, second tube 1048 can also include corrugations. The lumen (not labeled) defined by second tube 1048 can include a divider 1030, which divider acts to divide the lumen into multiple fluid pathways, e.g., with one pathway for fluid delivery and another pathway for fluid withdrawal, e.g., in a catheter-type application. A device can be a single- or multiple-lumen device; lumens can be coaxial, although this is not a rule or requirement. A device can be configured as a coaxial catheter, for example.

A device can include a first straight region 1026, as well as curved region 1024, and a second straight region 1023. A device can also include conduit 1031, which conduit can be used to contain a surgical implement (e.g., irrigator, optical instrument, illuminator, control/guide wire, and the like). Conduit 1031 can also be used to deliver or withdraw fluid; conduit 1031 can also be a hydraulic conduit that contains fluid that is in turn acted on to effect bending (or straightening) of the device.

Although not shown in FIG. 1, a device can include one or more guide/control wires that are used to steer the device. A wire can be used to pull on a portion of a device so as to effect bending (or straightening). A wire can also be used to effect rotation of a portion of the device. Such a wire can be disposed within conduit 1031.

A device can also include an aperture 1033 formed along its length. Fluid can be delivered and/or withdrawn through the aperture; it is not a requirement that fluid be delivered/withdrawn through the distal end of a device according to the present disclosure, as fluid can be delivered/withdrawn at one or more locations (e.g., apertures) along the length of the device. In this way, a device can be inserted into a patient with the insertion being facilitated by a feature (e.g., a point or cutting tip) of the distal end, and following insertion, fluid can be delivered at a location along the device that is not at the distal end of the device.

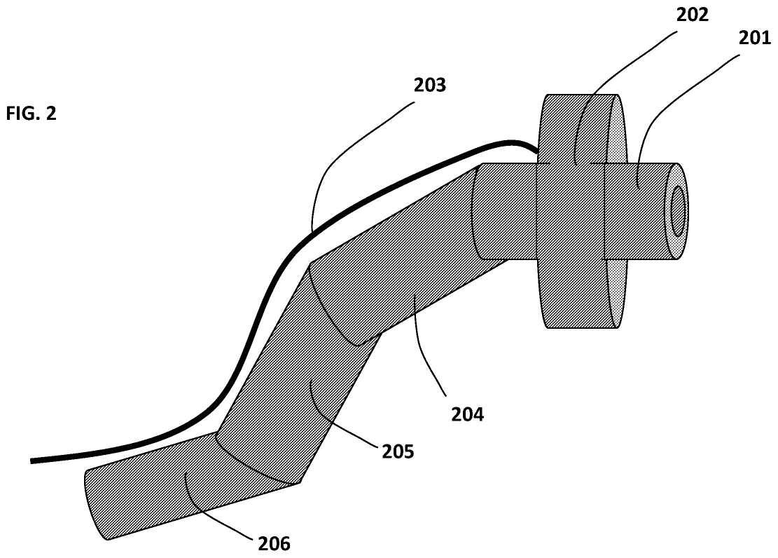

As shown in FIG. 2, a device can include multiple segments 201, 204, 205, and 206. A pull wire 203 can be connected to the device, e.g., to collar 202, to afford the user control over bending and straightening the device. It should be understood that the segments are shown for illustrative purposes only, as a device can be formed of a single set of flexible tubes, as opposed to a device formed of multiple segments connected to one another. A pull wire can be used to pull (or push), which allows a user to bend or straighten the device. A wire (or other element) can also be used to rotate/twist some or all of the device.

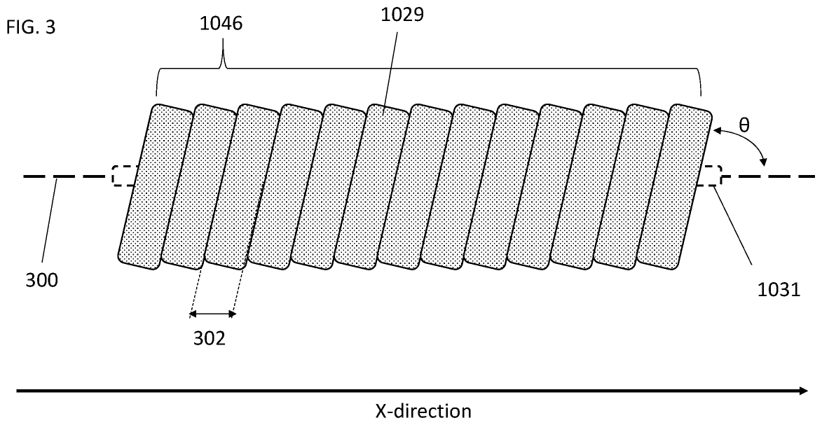

FIG. 3 provides a view of an exemplary device. As shown, first tube 1046 can include a plurality of (parallel) corrugations 1029 along its length. As shown, the corrugations can be disposed at an angle θ from major axis 300 of the first tube. Angle θ can be from −90 degrees to +90 degrees, preferably −45 degrees to +45 degrees. A corrugation can have a width 302. Corrugation width 302 can be variable along the length of tube 1046 (in the x-direction), but can also be constant. The pitch of the corrugations can be, e.g., from about 0.5 mm to about 5 mm, from about 1 mm to about 4.5 mm, from about 1.5 mm to about 4.0 mm, from about 2.0 mm to about 3.5 mm, or even about 2.5 mm. The corrugation pitch can be constant along the length of the tube 1046 (along the x-direction), but this is not a requirement, as the pitch can vary along the length of tube 1046 (in the x-direction).

The width and/or pitch and/or height of corrugations can be varied along the length of a tube so as to modulate the rigidity of the tube at different locations along the tube's length. As an example, the width, height, and pitch of corrugations can be modulated such that the tube is relatively bendable around the middle of the tube and is relatively stiff at the ends of the tube. A tube can also be telescoped (i.e., expanded) so as to change the tube's length, as well as the tube's rigidity. Corrugations can be formed so that the tube is bendable in only one direction, or so that the tube is preferentially bent in only one direction.

A component having corrugations that are oriented at an angle θ from major axis 300 can include any one or more of the other features described herein, e.g., conduit 1031, a pull wire, an aperture, and the like. A component can include corrugations that are all parallel to one another, but this is not a requirement, as a component can include two or more corrugations that are inclined at different angles (θ) from major axis 300.

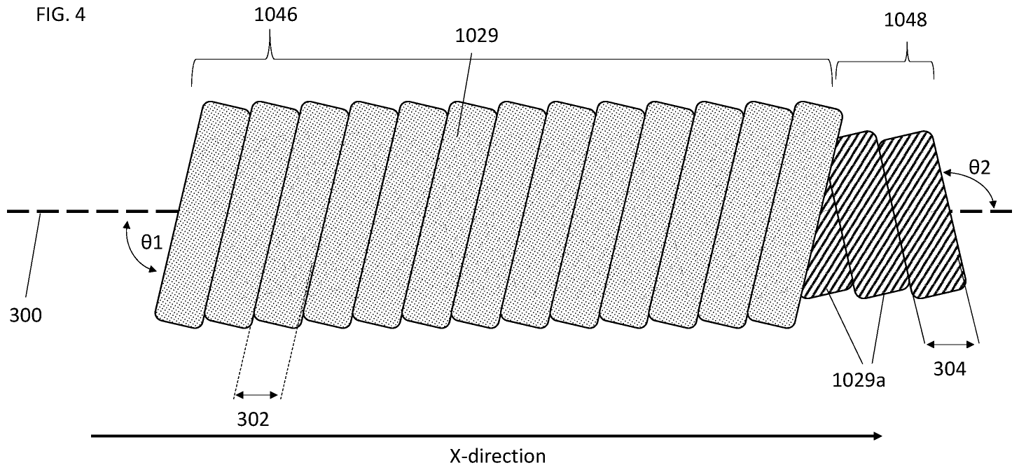

FIG. 4 provides a view of another embodiment of a component according to the present disclosure. As shown, tube 1046 can include a plurality of (parallel) corrugations 1029 along its length. As shown, the corrugations can be disposed at an angle θ1 from major axis 300 of the first tube. Angle θ1 can be from, e.g., −90 degrees to +90 degrees, preferably from −45 degrees to +45 degrees. A corrugation can have a width 302. Corrugation width 302 can be variable along the length of tube 1046 (in the x-direction), but can also be constant. As described elsewhere herein, the pitch of the corrugations in the first tube 1046 can be the same along the length of first tube 1046 (in the x-direction), but this is not a requirement, as the corrugation pitch can vary

Tube 1046 can enclose second tube 1048, which second tube can include corrugations 1029a, which corrugations are inclined at an angle θ2 from major axis 300 of the first tube. Angle θ2 can be from, e.g., −90 degrees to +90 degrees, preferably −45 degrees to +45 degrees. A corrugation can have a width 304. Corrugation width 304 can be variable along the length of tube 1048 (in the x-direction), but can also be constant. Corrugation width 304 can be, e.g, from about 0.5 mm to about 5 mm, or from about 1 mm to about 4.5 mm, or from about 1.5 mm to about 4.0 mm, or from about 2.0 mm to about 3.5 mm, or even from about 2.5 mm to about 3.0 mm. Similarly, the pitch of the corrugations in the second tube 1048 can be constant in the x-direction, but this is not a requirement, as the pitch of the corrugations in the second tube 1048 can vary in the x-direction. A sealed, evacuated space (described elsewhere herein) can be defined between first tube 1046 and second tube 1048. Corrugations 1029a can be parallel to one another, but individual corrugations 1029a can also be inclined at different angles from the major axis 300.

In addition, corrugations 1029a can also be of one or more spiral corrugations that, similar to the thread of a screw, extend around and along the length of second tube 1048. The pitch of such a spiral-type corrugation can be constant along the length of second tube 1048 (in the x-direction), but can also vary along the length of second tube 1048 (in the x-direction). One or both of first tube 1046 and second tube 1048 can comprise corrugations parallel to one another, and/or one or both of first tube 1046 and second tube 1048 can comprise corrugations that are spiral in configuration.

It should be understood that either one or both of first tube 1046 and second tube 1048 can be corrugated. It should also be understood that the corrugations in first tube 1046 can be inclined so as to “lean” in a direction that is different from the direction in which the corrugations in second tube 1048 “lean.” This is illustrated in FIG. 4, in which FIG. corrugations 1029 “lean” to the right, and corrugations 1029a “lean” to the left. It should be understood, however, that the first tube and the second tube can have corrugations that “lean” in the same direction. One of the first tube and the second tube can have corrugations that are perpendicular to the major axis 300, and the other of the first tube and the second tube can have corrugations that are non-perpendicular (whether parallel to one another or in spiral form) to the major axis 300.

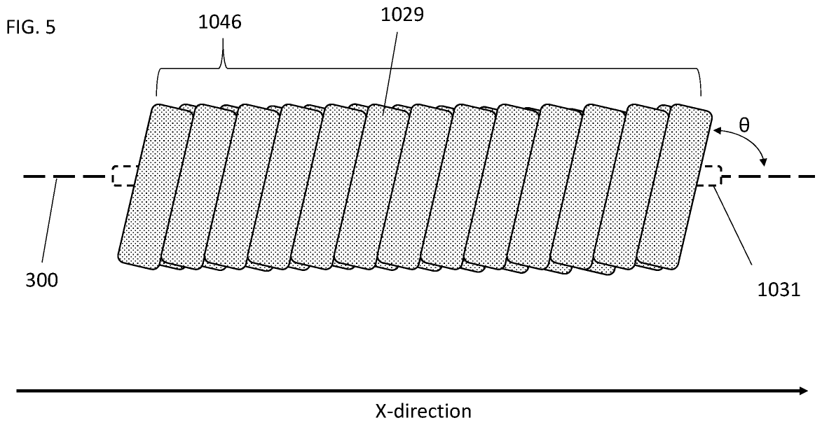

As shown in FIG. 5, corrugations 1029 can be of one or more spiral corrugations that, similar to the thread of a screw, extend around and along the length of first tube 1046. The pitch of such a spiral-type corrugation can be constant along the length of second tube 1046 (in the x-direction), but can also vary along the length of second tube 1046 (in the x-direction).

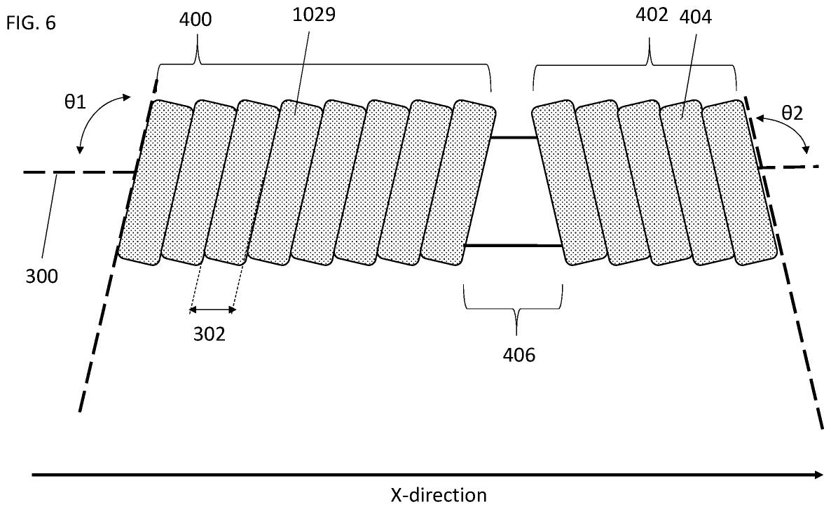

FIG. 6 provides a further exemplary embodiment. As shown, a tube (not labeled) can include a first region 400 having corrugations 1029 thereon that are angled at an angle θ1 relative to the centerline 300 of the tube. The tube can include a second region 402 having corrugations 404 thereon that are angled at an angle θ2 relative to the centerline 300. As shown, the tube can also include a region 400 that is uncorrugated, which region can connect corrugated regions 400 and 404.

The width of the corrugations (illustrated by 302) can be constant, but individual corrugations can have different widths and/or pitches. It should also be understood that corrugations can be parallel to one another, but a tube can include one or more regions where the corrugations are in spiral form, e.g., a region with a single spiral corrugation that runs along a region of the tube. The configuration shown in FIG. 4 can be utilized on an outer tube that encloses an inner tube, but can also be used on an inner tube that is enclosed within an outer tube.

### Embodiments

The following non-limiting embodiments are illustrative only and do not serve to limit the scope of the present disclosure or of the attached claims.

Embodiment 1. A thermally-insulated steerable device, comprising: an elongate body, the elongate body defining a major axis, the elongate body having a distal end, and the elongate body having a selectively steerable portion, the elongate body comprising an first tube; the elongate body comprising an second tube disposed within the first tube, the second tube defining a lumen therein, the lumen having a major axis, and the second tube and first tube defining a sealed insulating space of reduced pressure therebetween; a handle comprising an actuator; and a linkage in communication with the actuator, the linkage configured to effect a deflection of a portion of the elongate body from the major axis, a rotation of a portion of the elongate body about the major axis, or both.

Exemplary walls, sealing processes, and insulating spaces can be found in, e.g., US2018/0106414; US2017/0253416; US2017/0225276; US2017/0120362; US2017/0062774; US2017/0043938; US2016/0084425; US2015/0260332; US2015/0110548; US2014/0090737; US2012/0090817; US2011/0264084; US2008/0121642; US2005/0211711; WO/2019/014463; WO/2019/010385; WO/2018/093781; WO/2018/093773; WO/2018/093776; PCT/US2018/047974; WO/2017/152045; U.S. 62/773,816; and U.S. Pat. No. 6,139,571, the entireties of which documents are incorporated herein for any and all purposes.

A device can be configured such that the distal end of the device is configured for insertion into a subject. The distal end can be rounded, pointed, cupped, or otherwise shaped to support the needs of the user.

The device or a portion thereof is adapted to be inserted into a patient's body by way of a natural access (e.g., the nose, mouth, or rectum) or by a small incision in the body (e.g., into anatomical locations such as the pleural cavity, abdomen, or neck). In some embodiments, the device include a channel or other guide for permitting the passage of a surgical accessory implement (such as a cutting tool, a telescope, a manipulator, a secondary aspiration and irrigation device, etc.) into the surgical site.

A device can be configured for passage of fluid pumped from a remote fluid source ultimately to a surgical site. An irrigation control valve assembly operatively associated with the handle can allow the user to selectively modulate the flow of fluid through at least one of a first and a second fluid flow path. Fluid can be provided from a remote source, but a device according to the present disclosure can include a fluid source, e.g., a reservoir. In one embodiment, the device comprises a fluid flow control valve assembly that allows the user to selectively direct the flow of fluid. For example, the assembly can allow fluid to pass along a path that heats and/or cools the fluid and then passes the fluid to a surgical site to effect a therapeutic result. The assembly can allow fluid to pass along a path that sends the fluid directly to the surgical site without affirmative heating and/or cooling.

A device according to the present disclosure can include a heater and/or chiller configured to supply (or remove) heat from fluid passing within the device.

A device can include a temperature controller that receives fluid temperature information sensed by one or more temperature sensors disposed in or on the device.

In an alternative embodiment of the present invention, the device comprises an aspiration train, which train can valves, tubing, a source of pressure (and/or of negative or reduced pressure) and the like. The aspiration train permits the user to control the suction pressure to effect withdrawal of fluid or friable tissue from the surgical site.

In some embodiments, a material is disposed between the first tube and second tube, within the sealed insulating space between the tubes. The material can be in the form of a sheet, in woven form, in non-woven form, in fibrous form, in a porous form, in a perforated form, in a mesh form, and the like. The material can be disposed so as to reduce or eliminate physical contact between the first and second walls.

Embodiment 2. The device of Embodiment 1, wherein (a) the first tube comprises a corrugated region, (b) the second tube comprises a corrugated region, or both (a) and (b).

Corrugations can be annular; corrugations can also be spiral. Corrugations can be periodic, but this is not a requirement, as corrugations can be disposed in a non-periodic fashion. The first tube and the second tube can both be corrugated, though this is not a rule or requirement. The first tube and the second tube can bear the same type of corrugations (e.g., corrugations having the same height and the same period), but this is not a requirement. The second tube can have a corrugated region that is in at least partial register with a corrugated region of the first tube, but this also is not a requirement. A tube can include one, two, or more corrugated regions; corrugated regions can be separated from one another by non-corrugated regions.

Exemplary corrugations are provided in FIG. 3, FIG. 4, and FIG. 5. As described elsewhere herein, corrugations can be perpendicular to the major axis of a tube. This, however, is not a requirement, as corrugations can be non-perpendicular to the major axis of a tube. The corrugations can be parallel to one another, but can also be spiraled in configuration.

Embodiment 3. The device of Embodiment 2, wherein the corrugated region of the first tube overlies at least a portion of the corrugated region of the second tube.

Embodiment 4. The device of any one of Embodiments 1-3, wherein the linkage comprises a wire, a hinge, or both. A variety of linkages can be used. As one example, a linkage can include a wire running along (inside or outside) a portion of the device. The wire can be used to pull on a portion of the device so as to effect a bend. As one such example, a device can be constructed of plurality of connected segments—much like the bones of the finger—which segments can be pushed and/or pulled by action of the wire. A linkage can include one, two, or more segments that are actuated (e.g., by pushing, pulling, or twisting) by the user to effect bending of the device. An actuator can include one or more of a wheel, slider, trigger, thumbwheel, fingerwheel, bulb, spring, or other element configured to effect bending of a portion of the device. Segments can be arranged in a telescoping fashion.

As an example, a device can comprise first and second flexible tubes that define a sealed, insulating space therebetween. An actuator of the device can be used to exert a tension on a wire that runs along the device (e.g., within a channel and/or within one or more guides) so as to effect a bend in the device.

A device can be configured such that some or all of the device is twistable. A device can be constructed such that a portion of the device is rotatable. A device can be constructed such that the device comprises two or more portions that are independently bendable. A device can be constructed such that the device comprises two or more portions that are independently rotatable. A device can be constructed such that the device comprises two or more portions that are independently addressable, relative to one another. As an example, a device can include a first segment that is moveable in the X-Y plane, and a second segment that is independently moveable in the Y-Z plane.

Embodiment 5. The device of any one of Embodiments 1-4, wherein the linkage comprises a shape memory material.

Embodiment 6. The device of any one of Embodiments 1-5, wherein the linkage comprises a hydraulic element. As an example, a linkage can include a flexible conduit through which fluid is exerted so as to effect a bend and/or a straightening of the conduit. A linkage can also include a fluid-containing, flexible conduit within which the fluid can be acted on via positive or negative (reduced) pressure to effect bending and/or extension.

Embodiment 7. The device of any one of Embodiments 1-6, wherein the linkage comprises an electrically-actuated element.

Embodiment 8. The device of any one of Embodiments 1-7, wherein the linkage comprises a thermally-actuated element.

Embodiment 9. The device of any one of Embodiments 1-8, wherein comprising a vent defined by the second tube and the first tube communicating with the sealed insulating space to provide an exit pathway for gas molecules from the space, the vent being sealable for maintaining a vacuum within the sealed insulating space following evacuation of gas molecules through the vent, the distance between the first and second walls being variable in a portion of the insulating space adjacent the vent such that gas molecules within the insulating space are directed towards the vent by the variable-distance portion of the first and second walls during the evacuation of the insulating space, the directing of the gas molecules by the variable-distance portion of the first and second walls imparting to the gas molecules a greater probability of egress from the insulating space than ingress. An exemplary embodiment of such a vent is provided in U.S. Pat. No. 7,374,063, the entirety of which patent is incorporated herein by reference in its entirety for any and all purposes.

Embodiment 10. The device of any one of Embodiments 1-9, wherein the distal end comprises an opening formed therein. Such an opening can be used for, e.g., fluid delivery and/or fluid withdrawal.

Embodiment 11. The device of Embodiment 10, wherein the opening is in fluid communication with the lumen of the second tube.

Embodiment 12. The device of any one of Embodiments 1-11, wherein the lumen of the second tube is configured to communicate a fluid toward or away from the distal end of the elongate body. The lumen of the second tube can define within it one, two, or more fluid pathways. As an example, the lumen of the second tube can define a first pathway by which pathway fluid is delivered to a surgical or treatment site. The lumen of the second tube can also define (e.g., by way of an internal partition, tube, or other border) a second pathway by which pathway fluid is withdrawn.

For example, heated fluid can be delivered to a treatment site via a first fluid pathway within the lumen of the second tube. After the heated fluid is delivered to a treatment site, the fluid is withdrawn via negative pressure, along a second fluid pathway within the lumen of the second tube. Fluid can be withdrawn by a fluid pathway that is not within the lumen of the second tube, e.g., by a fluid pathway (such as a tube) that is disposed on or near the exterior of the steerable device.

Embodiment 13. The device of any one of Embodiments 1-12, further comprising a first jacket disposed about the first tube.

Embodiment 14. The device of Embodiment 13, wherein first jacket and first tube define a space therebetween.

Embodiment 15. The device of Embodiment 14, wherein the space is configured to configured to communicate a fluid toward or away from the distal end of the elongate body. In this way, the device can define a coaxial or multiple-lumen catheter arrangement.

Embodiment 16. The device any one of Embodiments 1-15, further comprising a third tube and a fourth tube disposed within the lumen of the second tube, the third tube and fourth tube defining a sealed insulating space of reduced pressure therebetween, the fourth tube defining a secondary lumen therein, the secondary lumen defining a major axis and the lumen optionally being coaxial with the lumen of the second tube.

Embodiment 17. The device of Embodiment 16, wherein (a) the third tube comprises a corrugated region, (b) the fourth tube comprises a corrugated region, or both (a) and (b). Corrugations can be annular and/or spiral; suitable corrugations are described elsewhere herein.

Embodiment 18. The device of Embodiment 17, wherein the corrugated region of the third tube overlies at least a portion of the corrugated region of the fourth tube.

Embodiment 19. The device of any one of Embodiments 16-18, wherein the secondary lumen is configured to communicate a fluid toward or away from the distal end of the elongate body.

Embodiment 20. The device of any one of Embodiments 1-19, further comprising a radiopaque marker. Such a marker can be used to locate one or more portions of the device before, during, or after usage, e.g., via x-ray or other imaging modality. A device can include other types of markers (e.g., a marker visible on ultrasound).

Embodiment 21. The device of any one of Embodiments 1-20, further comprising a fluidic coupler configured to place the device into fluid communication with a source of fluid.

Embodiment 22. The device of any one of Embodiments 1-21, further comprising a heater, chiller, or both. The heater/chiller can be used to change or maintain the temperature of a fluid before the fluid enters the device, while the fluid is within the device, or after the fluid exits the device.

Embodiment 23. A method, comprising inserting into a patient a portion of a device according to any one of Embodiments 1-22. As described elsewhere herein, inserting can be performed via an existing opening (e.g., nostril) of the patient, but can also be performed via incision.

Embodiment 24. A method, comprising operating the linkage of a device according to any of Embodiments 1-22 so as to effect a deflection of a portion of the elongate body from the major axis, a rotation of a portion of the elongate body about the major axis, or both.

Embodiment 25. A method, comprising communicating a fluid within a device according to any one of Embodiments 1-22.

Embodiment 26. The method of Embodiment 25, wherein the fluid is communicated so as to effect heating of a tissue proximate to the device.

Embodiment 27. The method of Embodiment 26, wherein the fluid is communicated to as to effect cooling of a tissue proximate to the device.

Embodiment 28. The method of any one of Embodiments 26-27, wherein the tissue is proximate to the distal end of the device.

Embodiment 29. The method of any one of Embodiments 25-28, wherein the tissue is in a disease state.

Embodiment 30. The method of Embodiment 29, wherein the disease state is cancer.

Embodiment 31. A thermally-insulated steerable device, comprising: an elongate body, the elongate body defining a major axis, the elongate body having a distal end, and the elongate body having a selectively steerable portion, the elongate body comprising an first tube; the elongate body comprising an second tube disposed within the first tube, the second tube defining a lumen therein, the lumen having a major axis, and the second tube and first tube defining a sealed insulating space of reduced pressure therebetween; and a linkage configured to effect a deflection of a portion of the elongate body from the major axis, a rotation of a portion of the elongate body about the major axis, or both.

One or both of the first tube and the second tube can include corrugations along at least part of the tube's length. Suitable corrugations (e.g., parallel corrugations, spiral corrugations, corrugations that are non-perpendicular to the major axis of the tube) are described elsewhere herein, e.g., in connection with FIGS. 3-5.

Embodiment 32. The thermally-insulated steerable device of Embodiment 31, wherein the lumen defines a channel for fluid delivery, for fluid withdrawal, or both.

Embodiment 33. The thermally-insulated steerable device of any one of Embodiments 31-32, further comprising a flow channel disposed within the lumen.

Embodiment 34. A thermally-insulated steerable device, comprising: a plurality of segments, a segment comprising an first tube; a segment comprising an second tube disposed within the first tube, the second tube defining a lumen therein, the lumen having a major axis, and the second tube and first tube defining a sealed insulating space of reduced pressure therebetween; and a linkage configured to effect relative motion of one segment relative to another segment.

One or both of the first tube and the second tube can include corrugations along at least part of the tube's length. Suitable corrugations (e.g., parallel corrugations, spiral corrugations, corrugations that are non-perpendicular to the major axis of the tube) are described elsewhere herein, e.g., in connection with FIGS. 3-5.

Relative motion can be, e.g., rotation, tilting, extension, contraction, or some combination thereof. Suitable linkages are described elsewhere herein, and can include, e.g., push/pull wires, springs, elastomers,

Embodiment 35. The thermally-insulated steerable device of Embodiment 34, wherein the lumens of at least two of the plurality of segments are in fluid communication with one another.

A device (e.g., a device according to of any one of Embodiments 34-35) can further include one or more joints configured to place the lumens of at least two of the plurality of segments into in fluid communication with one another, the one or more joints being flexible. It should be understood that the term “flexible” means bendable, but also includes articulated joints, e.g., joints that include hinges, pivots, and the like.

A device can include a jacket (e.g., a flexible material, such as a polymer) that covers the segments of the device so as to ease insertion and/or removal of the device from a subject. The jacket can be removable and/or disposable. The jacket can include one or more apertures so allow for fluid egress and/or ingress. An aperture in a jacket can be formed in register with an aperture formed in a segment so allow for egress (or ingress) of fluid.

Embodiment 36. A thermally-insulated device, comprising: a first tube, the first tube having an outer surface and defining a major axis, the outer surface of the first tube comprising a corrugated region that includes one or more corrugations, (a) the one or more corrugations defining a corrugation axis defined along the portion of the corrugation that is at the maximum distance measured radially outward from the major axis of the first tube, the corrugation axis being at a corrugation angle that is non-perpendicular to the major axis of the first tube, or (b) the corrugated region comprising a helical corrugation; and a second tube, the second tube having an outer surface and a major axis and the second tube being disposed within the first tube, and the second tube and first tube defining a sealed insulating space of reduced pressure therebetween.

Embodiment 37. The device of Embodiment 36, wherein the outer surface of the second tube comprises a corrugated region that includes one or more corrugations, (a) the one or more corrugations defining a corrugation axis defined along the portion of the corrugation that is at the maximum distance measured radially outward from the major axis of the second tube, the corrugation axis being at a corrugation angle that is non-perpendicular to the major axis of the second tube, or (b) the corrugated region of the second tube comprising a helical corrugation

Embodiment 38. The device of Embodiment 37, wherein the one or more corrugations of the second tube run in a direction different from a direction in which the one or more corrugations of the first tube run.

Embodiment 39. The device of any one of Embodiments 36-38, wherein the corrugated region of the first tube comprises a helical corrugation.

Embodiment 40. The device of any one of Embodiments 36-38, wherein the corrugated region of the first tube comprises one or more corrugations defining a corrugation axis defined along the portion of the corrugation that is at the maximum distance measured radially outward from the major axis of the first tube, the corrugation axis being at a corrugation angle that is non-perpendicular to the major axis of the first tube.

Embodiment 41. The device of any one of Embodiments 36-38, wherein the corrugated region of the first tube comprises corrugations having different widths, pitches, or both.

Embodiment 42. The device of Embodiment 37, wherein the corrugated region of the first tube comprises a helical corrugation.

Embodiment 43. The device of Embodiment 37, wherein the corrugated region of the first tube comprises one or more corrugations defining a corrugation axis defined along the portion of the corrugation that is at the maximum distance measured radially outward from the major axis of the first tube, the corrugation axis being at a corrugation angle that is non-perpendicular to the major axis of the first tube.

Embodiment 44. The device of Embodiment 37, wherein the corrugated region of the first tube comprises corrugations having different widths, pitches, or both.

Embodiment 45. The device of any one of Embodiments 36-44, further comprising a vent defined by the second tube and the first tube communicating with the sealed insulating space to provide an exit pathway for gas molecules from the space, the vent being sealable for maintaining a vacuum within the sealed insulating space following evacuation of gas molecules through the vent, the distance between the first and second walls being variable in a portion of the insulating space adjacent the vent such that gas molecules within the insulating space are directed towards the vent by the variable-distance portion of the first and second walls during the evacuation of the insulating space, the directing of the gas molecules by the variable-distance portion of the first and second walls imparting to the gas molecules a greater probability of egress from the insulating space than ingress.

Embodiment 46. A method, comprising bending a device according to any one of Embodiments 36-45.

Embodiment 47. A method, comprising: with a device according to any one of Embodiments 36-45, communicating a fluid within the second tube.

1. A thermally-insulated steerable device, comprising:
an elongate body, the elongate body defining a major axis, the elongate body having a distal end, and the elongate body having a selectively steerable portion,
the elongate body comprising a first tube;
the elongate body comprising a second tube disposed within the first tube,
the second tube defining a lumen therein, the lumen having a major axis, and
the second tube and first tube defining a sealed insulating space of reduced pressure therebetween;
a handle comprising an actuator; and
a linkage in communication with the actuator, the linkage configured to effect a deflection of a portion of the elongate body from the major axis, a rotation of a portion of the elongate body about the major axis, or both.

2. The device of claim 1, wherein (a) the first tube comprises a corrugated region, (b) the second tube comprises a corrugated region, or both (a) and (b).

3. The device of claim 2, wherein the corrugated region of the first tube overlies at least a portion of the corrugated region of the second tube.

4. The device of claim 1, wherein the linkage comprises (i) a wire, a hinge, or both, (ii) a shape memory material, (iii) a hydraulic element, or any combination of (i), (ii), or (iii).

5. (canceled)

6. (canceled)

7. (canceled)

8. (canceled)

9. The device of claim 1, wherein comprising a vent defined by the second tube and the first tube communicating with the sealed insulating space to provide an exit pathway for gas molecules from the space, the vent being sealable for maintaining a vacuum within the sealed insulating space following evacuation of gas molecules through the vent, the distance between the first and second walls being variable in a portion of the insulating space adjacent the vent such that gas molecules within the insulating space are directed towards the vent by the variable-distance portion of the first and second walls during the evacuation of the insulating space, the directing of the gas molecules by the variable-distance portion of the first and second walls imparting to the gas molecules a greater probability of egress from the insulating space than ingress.

10. The device of claim 1, wherein the distal end comprises an opening formed therein.

11. The device of claim 10, wherein the opening is in fluid communication with the lumen of the second tube.

12. The device of claim 1, wherein the lumen of the second tube is configured to communicate a fluid toward or away from the distal end of the elongate body.

13. (canceled)

14. (canceled)

15. (canceled)

16. The device of claim 1, further comprising a third tube and a fourth tube disposed within the lumen of the second tube, the third tube and fourth tube defining a sealed insulating space of reduced pressure therebetween, the fourth tube defining a secondary lumen therein, the secondary lumen defining a major axis and the lumen optionally being coaxial with the lumen of the second tube.

17. The device of claim 16, wherein (a) the third tube comprises a corrugated region, (b) the fourth tube comprises a corrugated region, or both (a) and (b).

18. The device of claim 17, wherein the corrugated region of the third tube overlies at least a portion of the corrugated region of the fourth tube.

19. The device of claim 16, wherein the secondary lumen is configured to communicate a fluid toward or away from the distal end of the elongate body.

20. The device of claim 1, further comprising a radiopaque marker; a fluidic coupler configured to place the device into fluid communication with a source of fluid; a heater, chiller, or both; or any combination thereof.

21. (canceled)

22. (canceled)

23. (canceled)

24. A method, comprising operating the linkage of a device according to claim 1 so as to effect a deflection of a portion of the elongate body from the major axis, a rotation of a portion of the elongate body about the major axis, or both.

25. (canceled)

26. (canceled)

27. (canceled)

28. (canceled)

29. (canceled)

30. (canceled)

31. A thermally-insulated steerable device, comprising:
an elongate body, the elongate body defining a major axis, the elongate body having a distal end, and the elongate body having a selectively steerable portion,
the elongate body comprising an first tube;
the elongate body comprising an second tube disposed within the first tube,
the second tube defining a lumen therein, the lumen having a major axis, and
the second tube and first tube defining a sealed insulating space of reduced pressure therebetween;
a linkage configured to effect a deflection of a portion of the elongate body from the major axis, a rotation of a portion of the elongate body about the major axis, or both.

32. The thermally-insulated steerable device of claim 31, wherein the lumen defines a channel for fluid delivery, for fluid withdrawal, or both.

33. The thermally-insulated steerable device of claim 31, further comprising a flow channel disposed within the lumen.

34. A thermally-insulated steerable device, comprising:
a plurality of segments, a segment comprising an first tube; a segment comprising an second tube disposed within the first tube, the second tube defining a lumen therein, the lumen having a major axis, and the second tube and first tube defining a sealed insulating space of reduced pressure therebetween; and a linkage configured to effect relative motion of one segment relative to another segment.

35. The thermally-insulated steerable device of claim 34, wherein the lumens of at least two of the plurality of segments are in fluid communication with one another.

36. The thermally insulated-steerable device of claim 34, further comprising one or more joints configured to place the lumens of at least two of the plurality of segments into in fluid communication with one another, the one or more joints being flexible.
